(19) United States
(12) Patent Application Publication
(10) Pub. No.: US 2006/0122579 A1
(43) Pub. Date: Jun. 8, 2006

(54) Treatment apparatus including stored treatment protocols, and associated method

(76) Inventor: Maurice A. Pisciottano, Venetia, PA (US)

Correspondence Address: Brij K. Agarwal;Eckert Seamans Cherin & Mellott, LLC, 44th Floor, 600 Grant Street, Pittsburgh, PA, 15219, US

(21) Appl. No.: 10/984,421

(22) Filed: Nov. 8, 2004

(51) Int. Cl. A61B 17/00

(52) U.S. Cl. 606/1

(57) ABSTRACT

An improved treatment apparatus includes a processor apparatus, an input apparatus, and an output apparatus having a therapeutic appliance, with the processor apparatus having a plurality of treatment protocols stored therein, and with the treatment protocols being usable in conjunction with the therapeutic appliance to provide treatment to a recipient. When the exemplary device is employed in conjunction with chiropractic science, each protocol may include, for instance, a number of treatment positions where the therapeutic appliance will be applied to the recipient, and may additionally include a number of predetermined operating parameters for each of the treatment positions. The protocol may alternatively and/or additionally include a specification of a particular configuration of the therapeutic appliance, such as including one or more implements that are desired to be employed in conjunction with performing the protocol. The treatment apparatus can output a plurality of indicators that can be selected by a practitioner and that are representative of anatomical locations and/or related afflictions. The indicators are associated with particular treatment protocols. Upon selecting an indicator, the device obtains from memory the treatment protocol and outputs a map of the various treatment positions. The protocol also includes specifications for various operating parameters for each of the treatment positions.

### BACKGROUND OF THE INVENTION

1. Field of the Invention

The invention relates generally to treatment apparatuses and, more particularly, to a treatment apparatus having a number of treatment protocols stored therein, and an associated method.

2. Description of the Related Art

Numerous different types of treatment apparatuses are known. Examples of such treatment apparatuses would include chiropractic adjustment equipment, ultrasound equipment, iontophoresis equipment, and the like, in addition to numerous other types of equipment. Such treatment apparatuses typically are employed to perform some type of an action on a human or animal recipient. Such actions can include, for example and without limitation, actions that are therapeutic in nature, actions that supply things to the recipient, actions that take measurements, and numerous other types of actions.

The use of some treatment apparatuses can be extremely complex based upon the complexity of the desired action and the complexity of the equipment, as well as other factors. For instance, the equipment may be capable of numerous types of treatments, such as might include treatments to different regions of the body, treatments to provide various types of therapies in response to various types of problems, and the like. A treatment apparatus may, for example, may be operable in accordance with a number of operating parameters, one or more of which can be varied by a user. Equipment can additionally or alternatively include a number of implements that may be employed in various circumstances. While known treatment apparatuses have been generally effective for their intended purposes, such treatment apparatus have not, however, been without limitation.

Depending upon the configuration of the treatment apparatus, the nature of the actions desired to be performed using the treatment apparatus, and other factors, the use of such treatment apparatus can require significant skill, experience, and judgment from the practitioner. This is the case in, for example, the field of chiropractic science and chiropractic treatment due to the complexity of the human body, the wide variety of chiropractic procedures that can be performed on the body, and the complexity of each such treatment, as well as other factors. It thus would be desirable to provide a treatment apparatus and an associated method that enable complex treatments to be provided without requiring the high level of experience, skill, and judgment that otherwise might be required to provide such complex treatments. Such treatment apparatus and method desirably could be employed in conjunction with chiropractic science or in other fields.

### SUMMARY OF THE INVENTION

An improved treatment apparatus includes a processor apparatus, an input apparatus, and an output apparatus having a therapeutic appliance, with the processor apparatus having a plurality of treatment protocols stored therein, and with the treatment protocols being usable in conjunction with the therapeutic appliance to provide treatment to a recipient. When the exemplary device is employed in conjunction with chiropractic science, each protocol may include, for instance, a number of treatment positions where the therapeutic appliance will be applied to the recipient, and may additionally include a number of predetermined operating parameters for each of the treatment positions. The protocol may alternatively and/or additionally include a specification of a particular configuration of the therapeutic appliance, such as including one or more implements that are desired to be employed in conjunction with performing the protocol. The treatment apparatus can output a plurality of indicators that can be selected by a practitioner and that are representative of anatomical locations and/or related afflictions. The indicators are associated with particular treatment protocols. Upon selecting an indicator, the device obtains from memory the treatment protocol and outputs a map of the various treatment positions. The protocol also includes specifications for various operating parameters for each of the treatment positions.

Accordingly, an aspect of the invention is to provide an improved treatment apparatus having a plurality of stored treatment protocols that can be employed by a practitioner in performing an action with the treatment apparatus.

Another aspect of the invention is to provide on a treatment apparatus a plurality of treatment protocols that simplify operation of the treatment apparatus by a practitioner.

Another aspect of the invention is to provide an improved treatment apparatus having a user interface that enables a practitioner to interact with predetermined treatment protocols to perform actions with the treatment apparatus.

Another aspect of the invention is to provide an improved treatment apparatus that can instruct a practitioner where to apply a therapeutic appliance and that can control the adjustment of various operating parameters of the therapeutic appliance.

Another aspect of the invention is to provide an improved method of directing a number of applications of a therapeutic appliance of a treatment apparatus, wherein the treatment apparatus includes a plurality of treatment protocols stored therein.

Another aspect of the invention is to provide an improved machine readable medium that provides instructions that cause the treatment apparatus to enable a user to perform a treatment action with the treatment apparatus.

Accordingly, an aspect of the invention is to provide an improved treatment apparatus, the general nature of which can be stated as including a processor apparatus including a processor and a memory, an input apparatus, and an output apparatus including a display and a therapeutic appliance. The display is adapted to display a plurality of indicators. The memory has a plurality of treatment protocols stored therein, with each treatment protocol of at least a portion of the plurality of treatment protocols being associated with at least a first indicator from among the plurality of indicators. Responsive to detecting a selection input with respect to an indicator from among the plurality of indicators, the processor is adapted to retrieve from the memory from among the plurality of treatment protocols a treatment protocol that is associated with the indicator, and to output to the display an output representative at least in part of at least a portion of the treatment protocol. The processor and the therapeutic appliance are connected together and, responsive to detecting a predetermined input, the processor is adapted to actuate the therapeutic appliance to operate in accordance with at least a portion of the treatment protocol.

Another aspect of the invention is to provide an improved method of directing a number of applications of a therapeutic appliance of a treatment apparatus, the treatment apparatus including a processor apparatus, an input apparatus, and an output apparatus. The processor apparatus includes a memory having a plurality of treatment protocols stored therein. The output apparatus includes a display and the therapeutic appliance. The general nature of the method can be stated as including outputting on the display a plurality of indicators, detecting a selection input with respect to an indicator of the plurality of indicators and, responsive to detecting a selection input, retrieving from the memory a treatment protocol of the plurality of treatment protocols that corresponds with the indicator. The treatment protocol includes a number of treatment positions, with each treatment position of at least a portion of the number of treatment positions being a position on a recipient where an operation of the therapeutic appliance is specified by the protocol to occur. The method further includes outputting on the display an output including at least a first portion of the treatment protocol.

Another aspect of the invention is to provide an improved machine readable medium that provides instructions which, when executed on a treatment apparatus of a type including a processor apparatus, an input apparatus, and an output apparatus, with the output apparatus including a display and a therapeutic appliance, cause the treatment apparatus to perform operations. The general nature of the operations can be stated as including outputting on the display a plurality of indicators, detecting a selection input with respect to an indicator of the plurality of indicators and, responsive to detecting a selection input, retrieving from a memory a treatment protocol from among a plurality of treatment protocols. The treatment protocol corresponds with the indicator and includes a number of treatment positions, with each treatment position of at least a portion of the number of treatment positions being a position on a recipient where an operation of the therapeutic appliance is specified by the protocol to occur. The operations further include outputting on the display an output including at least a first portion of the treatment protocol.

### BRIEF DESCRIPTION OF THE DRAWINGS

A full understanding of the invention can be gained from the following Description of the Preferred Embodiment when read in conjunction with the accompanying drawings in which:

FIG. 1 is a perspective view of a treatment apparatus in accordance with the invention;

FIG. 2 is a schematic depiction of the treatment apparatus of FIG. 1;

FIG. 3 is an exemplary output on a display of the treatment apparatus of FIG. 1I;

FIG. 4 is a schematic depiction of a treatment protocol stored on the treatment apparatus of FIG. 1;

FIG. 5 is an exemplary flowchart depicting at least a portion of a method in accordance with the invention;

FIG. 6 is an exemplary output of at least a portion of a treatment protocol of the treatment apparatus of FIG. 1; and

FIG. 7 is another exemplary output of at least a portion of the treatment protocol of the treatment apparatus of FIG. 1.

Similar numerals refer to similar parts throughout the specification.

### DESCRIPTION OF THE PREFERRED EMBODIMENT

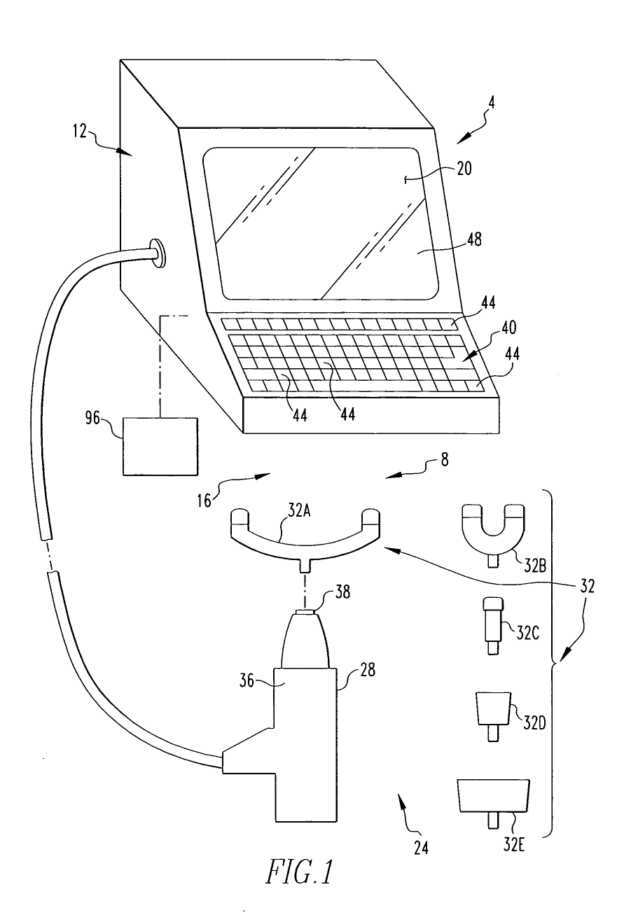

An improved treatment apparatus 4 in accordance with the invention is depicted generally in FIG. 1. The treatment apparatus 4 includes an input apparatus 8, a processor apparatus 12, and an output apparatus 16. The exemplary treatment apparatus 4 can be used, for example, in performing chiropractic treatments, although it is understood that the treatment apparatus 4 likely could be used for other treatments, and it is further understood that the teachings herein could be applied to other equipment employed in other types of treatments, without limitation. As will be set forth in greater detail below, the treatment apparatus 4 is advantageously configured to enable treatments to be provided therewith generally without requiring the extensive skill, experience, and judgment that might be required in providing similar treatments with other types of equipment.

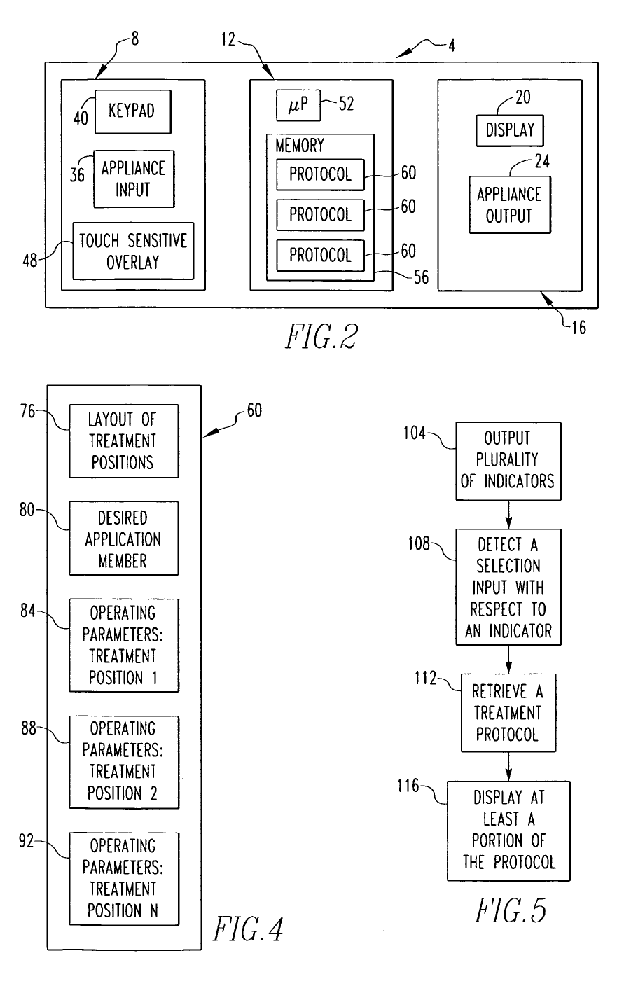

As can be understood from FIGS. 1 and 2, the output apparatus 16 includes a display 20 and a therapeutic appliance 24. As will be set forth in greater detail below, the display 20 provides output that directs a practitioner in positioning the therapeutic appliance 24 at various treatment positions in order to provide a treatment to a recipient. The exemplary therapeutic appliance 24 is a reciprocating device that, for example, can be used in performing spinal adjustments and in providing other therapeutic applications. Depending upon the types of treatment desired, it is understood that the therapeutic appliance 24 could be replaced with other types of therapeutic appliances such as, for example and without limitation, ultrasound transceivers or other devices without departing from the concept of the invention.

The exemplary therapeutic appliance 24 that is depicted in FIGS. 1 and 2 includes an actuator 28 upon which can be mounted any of a number of application members 32. The exemplary therapeutic appliance 24 additionally includes a sensor 36 that is part of the input apparatus 8, as will be described in greater detail below. As a general matter, the processor apparatus 12 is configured to detect input from the input apparatus 8 and to provide output to the output apparatus 16.

The actuator 28 is, in the present example, an electrically operated device that provides a reciprocating motion to whichever application member 32 is mounted at a tip 38 of the actuator 28. The actuator 28 operates according to a number of operating parameters such as frequency, intensity and duration. As employed herein, the expression of “a number of” and variations thereof shall refer broadly to any nonzero quantity, including a quality of one. The frequency operating parameter can, for example, refer to the frequency of reciprocation of the actuator 28. The intensity operating parameter can, for example, refer to the energy of each reciprocation. The duration operating parameter can refer to the total quantity of reciprocations of the actuator 28. In the present example, all three of these operating parameters can be varied. Other operating parameters may be provided. The therapeutic appliance 24 may, for instance, be a device such as that described generally in U.S. Pat. No. 6,539,328 to Cremonese, et al. the disclosures of which are incorporated by reference herein.

As can be seen from FIG. 1, the exemplary depicted application members 32 include a wide-prong application member 32A, a narrow prong application member 32B, a single prong application member 32C, a small block-type application member 32D, and a large block-type application member 32E. These various application member 32 can be interchangeably mounted to the actuator 28. During an exemplary reciprocating operation of the actuator 28, the application member 32 that is mounted on the tip 38 of the actuator 28 is reciprocated by the actuator 28 to provide a therapeutic action if brought into contact with a recipient at a desired location.

The input apparatus 8 includes a keypad 40 that includes a plurality of keys 44, a touch sensitive overlay 48 that is disposed adjacent the display 20, and the aforementioned sensor 36 that is mounted to the actuator 28. The touch sensitive overlay 48 is any of a wide variety of known devices and is configured to provide an input to the processor apparatus 12 upon being touched, for example, by a practitioner. The particular input provided by the touch sensitive overlay 48 in response to a touch input varies in a known fashion based upon the location of the touch input on the touch sensitive overlay 48.

The sensor 36 can be any of a wide variety of sensors that detect motion and/or vibration, and potentially can be a piezoelectric sensor. The sensor 36 additionally or alternatively could be a load sensor that detects loading on the application member 32.

When the application member 32 experiences a load of, for example, six pounds, such as if the application member 32 were applied to a recipient and the actuator 28 pressed toward the recipient with a force of six pounds, the therapeutic appliance 24 sends a predetermined input to the processor apparatus 12 indicating that the therapeutic appliance 24 is properly positioned on the recipient and is ready for initiation of an operation of the actuator 28.

As can be understood from FIG. 2, the processor apparatus 12 includes a processor 52 and a memory 56. The processor 52 may be, for example and without limitation, a microprocessor (μP) or any other type of processor. The memory 56 is cooperable with the processor 52 and may be any of a variety of internal and/or external storage media including RAM, ROM, EPROM, EEPROM, and/or the like. The memory 56 includes a number of routines stored therein that are not expressly depicted in FIG. 2. The memory 56 additionally, and in accordance with the invention, includes a plurality of protocols 60 stored therein which, in the present exemplary embodiment, are treatment protocols. As will be set forth in greater detail below, each protocol 60 includes various specifications for the conduct of a treatment on a recipient using the treatment apparatus 4.

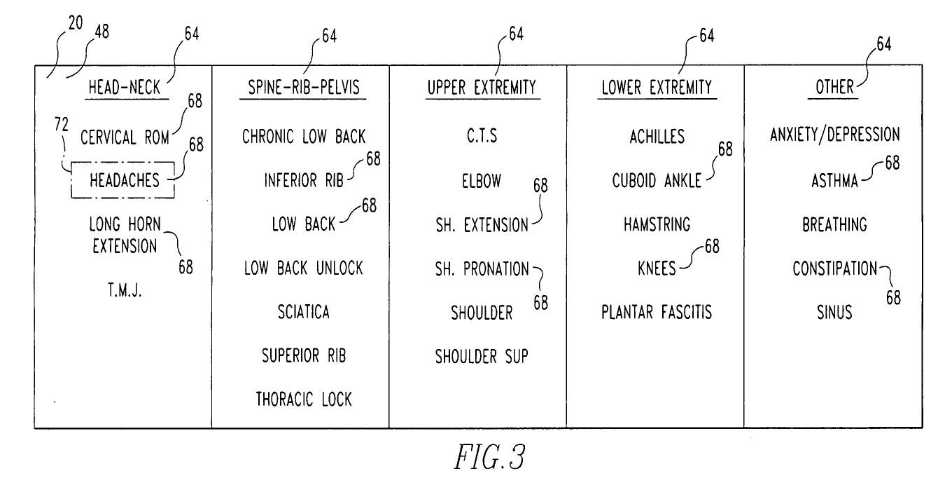

In using the treatment apparatus 4, the display 20 outputs a plurality of anatomical categories 64 which, in the present exemplary embodiment describe major physical regions or systems such as, for example, “Head-Neck”,“Spine-Rib-Pelvis”, and the like, as are depicted in FIG. 3. The display 20 also outputs a plurality of indicators 68 that represent anatomical locations and/or related afflictions. Generally each indicator 68 is associated with an anatomical category 64, it being noted that some of the indicators 68 are associated with the anatomical category 64 of “Other”. An association between the indicators 68 and the anatomical categories 64 is visually depicted by providing one of the anatomical categories 64 as a heading, and by having the associated indicators 68 depicted below the anatomical category 64. Other ways of depicting the association will be apparent.

Each of the indicators 68 is associated with at least one of the protocols 60. By providing an input such as a selection input with respect to one of the indicators 68, a corresponding protocol 60 is retrieved from the memory 56 and is generally made operative on the treatment apparatus. A selection input can, for example, be provided with respect to one of the indicators 68 by, for instance, touching the touch sensitive overlay 48 at a location that corresponds with the position at which the desired indicator 68 is depicted on the display 20, such as is indicated by the box 72 in FIG. 3. The exemplary box 72 depicts an entry of a selection input with respect to the indicator 68 that is labeled “Headaches”. Upon detecting the selection input with respect to the indicator 68 labeled “Headaches”,the processor 52 retrieves from the memory 56 the protocol 60 that corresponds with the “Headaches” indicator 68. The retrieved protocol 60 is then made operative on the treatment apparatus.

An exemplary protocol 60, such as the protocol 60 that is associated with the indicator 68 labeled “Headaches”, is schematically depicted in FIG. 4. The protocol 60 includes a layout 76 of the locations of a number of treatment positions specified by the protocol 60. The protocol 60 additionally includes a desired application member 80 that is specified for use in performing the protocol 60. The protocol 60 additionally includes a number of specified operating parameters for each treatment position that is included in the layout of treatment positions 76. For example, the protocol 60 includes a specification of operating parameters for Treatment Position 1 (shown by the numeral 84), a specification of operating parameters for Treatment Position 2 (shown by the numeral 88), and a specification of operating parameters for Treatment Position n (shown by the numeral 92). The specification of operating parameters for Treatment Position n is intended to refer to specifications of operating parameters for each of the additional treatment positions of the protocol 60. As will be described in greater detail below, the layout of treatment positions 76 and the desired application member 80 are output to the display 20 and are depicted for the practitioner. The practitioner can follow the output on the display 20 to position the therapeutic appliance 24 at each of the specified treatment positions in the specified sequence, with the operating parameters being set for each treatment position by the various stored operating parameters 84, 88, and 92 of the protocol 60.

It is noted that the routines and protocols 60 can be stored on a machine readable medium 96 along with other instructions which, if executed on a machine such as the treatment apparatus 4, will cause the treatment apparatus 4 to execute instructions such as are described herein in accordance with the method of the invention which will be set forth in greater detail below. The machine readable medium 96 may have been employed to initially load the protocols 60, routines, and other instructions in the memory 56. Thereafter, the memory 56 can itself serve as a machine readable medium upon which instructions are stored. It is noted that the machine readable medium 96 can be any of a wide variety of media such as, for example, various ROM devices, tape, various types of memory, and the like without limitation.

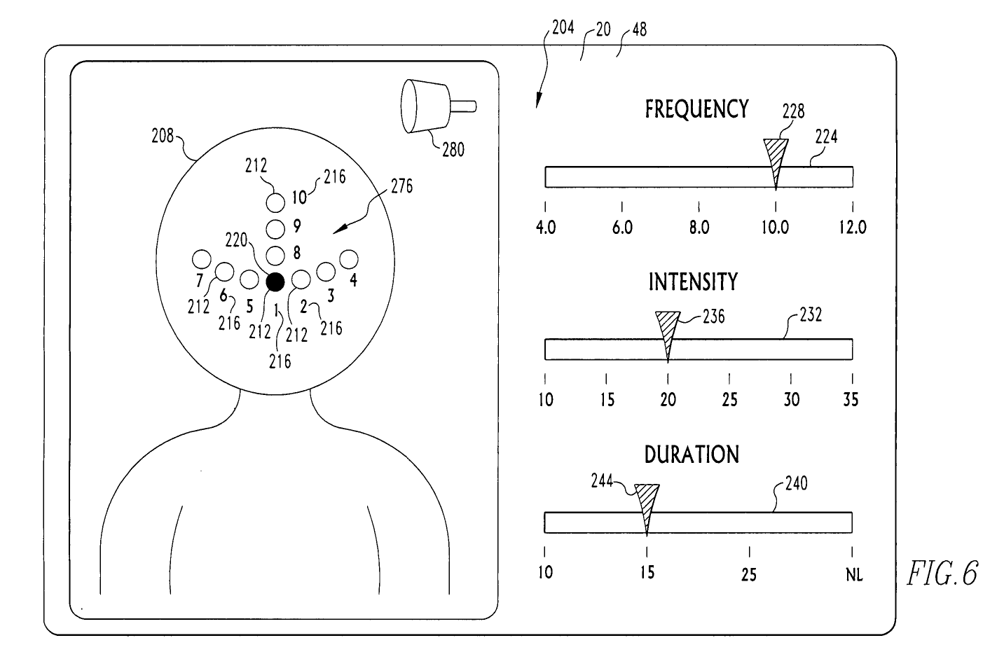

The method of the invention can be generally described as follows in reference to FIG. 5. The indicators 68 are output, such as at the numeral 104, on the display 20. The processor apparatus 12 detects, as at the numeral 108, a selection input with respect to one of the indicators 68. In response to detecting the selection input, the processor 52 retrieves, as at the numeral 112, from the memory 56 a protocol 60 that is associated with the selected indicator 68. At least a portion of the protocol 60 is then displayed, as at the numeral 116, on the display 120. A practitioner can thereafter interact with the treatment apparatus 4 with the protocol 60 being active thereon.

Upon activating the protocol 60 on the treatment apparatus 4, for example, the processor apparatus 12 may output to the display 20 a visual output 204 such as is depicted generally in FIG. 6. The visual output 204 includes, for example, a visual depiction of a map of treatment positions 276 that is representative of the layout of treatment positions 76 of the protocol 60. The visual output 204 can additionally include a visual depiction of an application member 280, which is representative of the stored desired application member 80 of the protocol 60.

The map of treatment positions 276 is output in a fashion superimposed over a depiction of an exemplary recipient 208 and includes a plurality of treatment positions 212 along with a corresponding sequence reference 216 for each such treatment position 212. That is, the exemplary treatment positions 212 indicate the positions at which the therapeutic appliance 24 will be applied, and the sequence references 216 describe the order of the treatment positions 212 for application by the therapeutic appliance 24.

The display 20 also depicts one of the treatment positions 212 as being a current treatment position 220. In the present example, the current treatment position 220 is indicated in FIG. 6 as being a filled circle at the treatment position 212 having the sequence reference 216 of “1”, while the other treatment positions 212 are indicated with empty circles. The current treatment position 220 could, for instance, be depicted by depicting the corresponding treatment position 212 in a color that is different than the color in which the other treatment positions 212 are depicted. Other possibilities will be apparent.

In accordance with the invention, the exemplary visual output 204 and FIG. 6 is depicted on the display 20 generally at the initiation of the protocol 60. The current treatment position 220 is depicted as the treatment position 212 that corresponds with the sequence reference 21 6 of “I”. The visual output 204 thus instructs the practitioner to apply the therapeutic appliance 24 while using the application member 32 that is shown in the depiction of the application member 280 to the position on the recipient 208 that is indicated by the current treatment position 220. Upon detecting a predetermined input, such as an input that results from pressing the application member 32 against the recipient 208 with the aforementioned force of six pounds, an operation of the therapeutic appliance 24 is initiated. As mentioned above, the protocol 60 includes specified operating parameters for each of the treatment positions 212. For the exemplary situation depicted in FIG. 6 wherein the current treatment position 220 is the treatment position 212 having the sequence reference 216 of “1”, the processor 52 sets the operating parameters of the therapeutic appliance 24 in accordance with the above-mentioned operating parameters for Treatment Position 1 which is denoted by the numeral 84. The exemplary operating parameters for Treatment Position 1 , shown at the numeral 84, include specifications of, for example, values for frequency, intensity, and duration. The operation of the therapeutic appliance 24 in the current operation, i.e., that corresponding with the current treatment position 220 being the treatment position 212 corresponding with the sequence reference 216 of “1”, is in accordance with the specified operating parameters for Treatment Position 1, as is shown at the numeral 84.

The exemplary visual output 204 additionally depicts these operating parameters. That is, the visual output 204 includes a visual depiction of the frequency parameter at 224, along with a cursor 228 which indicates the current setting of the frequency parameter 224. The intensity parameter is indicated at the numeral 232 and includes a cursor 236 that depicts the current setting of the intensity parameter 232. The duration parameter is depicted at the numeral 240 and includes a cursor 244 which visually depicts the current setting of the duration parameter 242. The depicted parameters 224, 232, and 240, along with the corresponding cursors 228, 236 and 244, provide to a practitioner a visual indication of the operating parameter values that are being employed in the current operation of the therapeutic appliance 24.

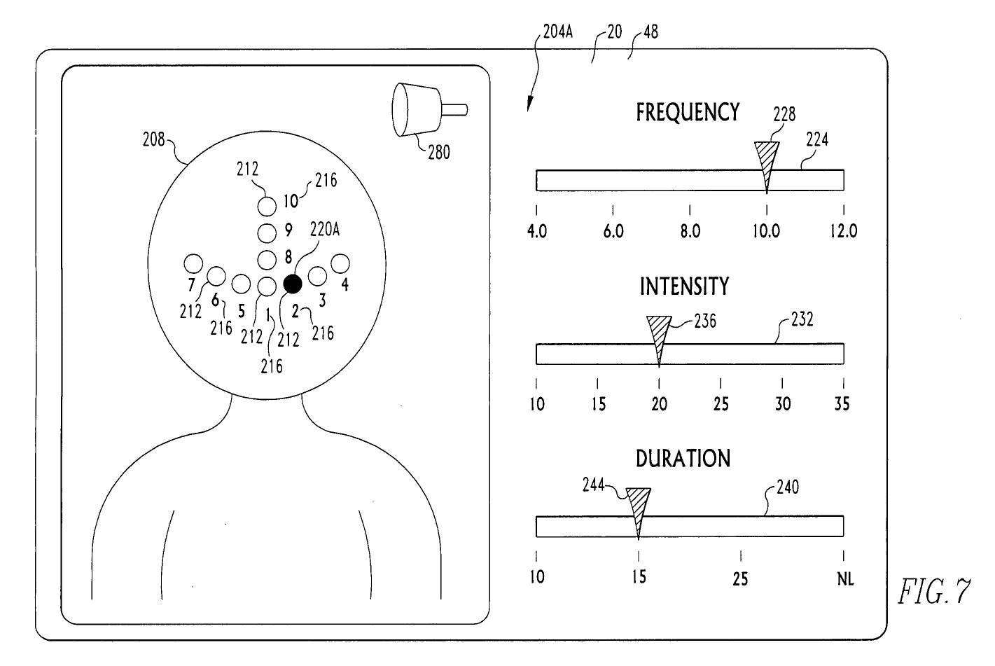

Once the current operation of the treatment apparatus 24 at the current treatment position 220 has been completed, the display 20 outputs an updated visual output 204A, such as is depicted generally in FIG. 7. FIG. 7 depicts the current treatment position 220A as now being the treatment position 212 having the sequence reference 216 of “2”. The operating parameters of frequency, intensity, and duration are similarly output. In the exemplary embodiment depicted herein, the values for the operating parameters of frequency, intensity, and duration are the same between the two treatment positions 212 having the sequence references 216 of “1” and “2”. It is noted, however, that in the exemplary embodiment depicted herein, operating parameters are specified for each treatment position 212, and the operating parameters need not stay the same for successive treatment positions 212.

It is noted that the depiction of the operating parameters 224, 232, and 240, along with their cursors, 228, 236, and 244, depict to the practitioner the current settings for such operating parameters. It is noted, however, that the practitioner can provide an override input with respect to any of the operating parameters, with the override input being representative of an alternate value for such operating parameter that is different than the value that is stored in the protocol 60. This enables the practitioner to depart from the protocol 60 if desired.

The treatment apparatus therefore advantageously enables a practitioner to select an indicator 68 which activates an associated protocol 60 on the treatment apparatus 4. The protocol 60 provides a visual indication of the various treatment positions 212 and corresponding sequence references 216 which detail the successive positions on the recipient 208 where the therapeutic appliance 24 will be applied in performing the protocol 60. The various operating parameters for each treatment position 212 are also stored in the protocol 60 and are used in setting the operating parameters of the therapeutic appliance 24 at each such treatment position 212. Advantageously, therefore, numerous treatments can be provided by the practitioner based upon the protocols 60 and the selection thereof based upon the display indicators 68 without requiring the high level of skill, experience, and judgment that would be required of the practitioner in the absence of protocols 60. That is, the protocols establish the treatment positions 212, the sequence references 216, and the various operating parameters for each such treatment position 212, and these are depicted to the practitioner, along with the depiction of the application member 280, which facilitates the provision of a treatment by the practitioner.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

What is claimed is:

1. A treatment apparatus comprising:
a processor apparatus including a processor and a memory;
an input apparatus;
an output apparatus including a display and a therapeutic appliance;
the display being adapted to display a plurality of indicators;
the memory having a plurality of treatment protocols stored therein, each treatment protocol of at least a portion of the plurality of treatment protocols being associated with at least a first indicator from among the plurality of indicators;
responsive to detecting a selection input with respect to an indicator from among the plurality of indicators, the processor being adapted to retrieve from the memory from among the plurality of treatment protocols a treatment protocol that is associated with the indicator, and to output to the display an output representative at least in part of at least a portion of the treatment protocol;
the processor and the therapeutic appliance being connected together and, responsive to detecting a predetermined input, the processor being adapted to actuate the therapeutic appliance to operate in accordance with at least a portion of the treatment protocol.

2. The treatment apparatus of claim 1 wherein the therapeutic appliance operates according to a number of operating parameters, at least one of the operating parameters being adjustable, the processor being adapted to adjust the at least one of the operating parameters on the therapeutic appliance in accordance with at least a portion of the treatment protocol.

3. The treatment apparatus of claim 2 wherein the at least one of the operating parameters includes at least one of frequency, duration, and intensity.

4. The treatment apparatus of claim 2 wherein, responsive to a predetermined input, the processor overrides the treatment protocol and adjusts the at least one of the operating parameters on the therapeutic appliance in accordance with at least a portion of the predetermined input.

5. The treatment apparatus of claim 1 wherein the treatment protocol includes a number of treatment positions, each treatment position of at least a portion of the number of treatment positions being a position on a recipient where an operation of the therapeutic appliance is specified by the protocol to occur, the output including a visual depiction of at least a portion of the number of treatment positions.

6. The treatment apparatus of claim 5 wherein the output includes a depiction of a plurality of treatment positions of the number of treatment positions, and wherein the output further includes an identification of at least one of the treatment positions of the plurality of treatment positions as being a current treatment position.

7. The treatment apparatus of claim 6 wherein each treatment position of at least a portion of the plurality of treatment positions is depicted in a first color, and wherein the identification of the at least one of the treatment positions includes a depiction of the at least one of the treatment positions in a second color that is different than the first color.

8. The treatment apparatus of claim 6 wherein the processor is adapted to detect a predetermined input indicative of an operation of the therapeutic appliance and, responsive to a detection of the predetermined input, the processor is adapted to output to the display an identification of another treatment position of the plurality of treatment positions as being a current treatment position.

9. The treatment apparatus of claim 6 wherein the output includes a visual depiction of a sequential order of the treatment positions of the plurality of treatment positions.

10. The treatment apparatus of claim 1 wherein the output apparatus is adapted to display as the plurality of indicators a plurality of identified anatomical locations.

11. The treatment apparatus of claim 10 wherein the output apparatus is adapted to output a plurality of anatomical categories, each anatomical category of at least a portion of the plurality of anatomical categories having associated therewith a number of anatomical locations of the plurality of anatomical locations, the display being adapted to output at least a first anatomical category of the at least a portion of the plurality of anatomical categories and the number of anatomical locations associated therewith in a fashion depicted an association therebetween.

12. The treatment apparatus of claim 1 wherein the therapeutic appliance includes a plurality of application members, the treatment protocol including a specification of a predetermined application member from among the plurality of application members, the output including a visual depiction of the predetermined application member.

13. The treatment apparatus of claim 1 wherein the input apparatus includes a touch sensitive overlay disposed adjacent the display, the touch sensitive overlay being adapted to detect the selection input as to the indicator by detecting a touch input at a position on the touch sensitive overlay corresponding with a position on the display where the indicator is depicted.

14. A method of directing a number of applications of a therapeutic appliance of a treatment apparatus, the treatment apparatus including a processor apparatus, an input apparatus, and an output apparatus, the processor apparatus including a memory having a plurality of treatment protocols stored therein, the output apparatus including a display and the therapeutic appliance, the method comprising:
outputting on the display a plurality of indicators;
detecting a selection input with respect to an indicator of the plurality of indicators;
responsive to said detecting a selection input, retrieving from the memory a treatment protocol of the plurality of treatment protocols that corresponds with the indicator, the treatment protocol including a number of treatment positions, each treatment position of at least a portion of the number of treatment positions being a position on a recipient where an operation of the therapeutic appliance is specified by the protocol to occur; and
outputting on the display an output including at least a first portion of the treatment protocol.

15. The method of claim 14, further comprising obtaining from the protocol a number of operating parameter settings for the treatment appliance, and outputting at least a first operating parameter setting of the number of operating parameter settings.

16. The method of claim 15, further comprising detecting a predetermined input and, responsive to said detecting a predetermined input, initiating an operation of the therapeutic appliance in accordance with the at least a first operating parameter setting.

17. The method of claim 15, further comprising detecting an override input with respect to the at least a first operating parameter setting, the override input being representative of an alternate value for the first operating parameter setting, detecting a predetermined input and, responsive to said detecting a predetermined input, initiating an operation of the therapeutic appliance in accordance with the alternate value.

18. The method of claim 14, further comprising outputting as the at least a first portion of the treatment protocol a visual depiction of a plurality of treatment positions of the number of treatment positions, and identifying at least one of the treatment positions of the plurality of treatment positions as being a current treatment position.

19. The method of claim 18, further comprising outputting a visual depiction of a sequential order of the plurality of treatment positions.

20. The method of claim 18, further comprising identifying each treatment position of at least a portion of the plurality of treatment positions with a first color, and identifying the current treatment position in a second color that is different than the first color.

21. The method of claim 18, further comprising detecting a predetermined input indicative of an operation of the therapeutic appliance and, responsive to said detecting a predetermined input indicative of an operation of the therapeutic appliance, outputting to the display an identification of another treatment position of the plurality of treatment positions as being a current treatment position.

22. The method of claim 14, further comprising displaying as the plurality of indicators a plurality of identified anatomical locations.

23. The method of claim 22, further comprising outputting a plurality of anatomical categories, each anatomical category of at least a portion of the plurality of anatomical categories having associated therewith a number of anatomical locations of the plurality of anatomical locations, and outputting a depiction of an association between at least a first anatomical category of the at least a portion of the plurality of anatomical categories and the number of anatomical locations associated therewith.

24. The method of claim 14 wherein the therapeutic appliance includes a plurality of application members, the treatment protocol including a specification of a predetermined application member from among the plurality of application members, and further comprising outputting a visual depiction of the predetermined application member.

25. The method of claim 14 wherein the input apparatus includes a touch sensitive overlay disposed adjacent the display, and further comprising detecting as the selection input a touch input at a position on the touch sensitive overlay corresponding with a position on the display where the indicator is depicted.

26. A machine readable medium that provides instructions which, when executed on a treatment apparatus of a type including a processor apparatus, an input apparatus, and an output apparatus, the output apparatus including a display and a therapeutic appliance, cause the treatment apparatus to perform operations comprising:
outputting on the display a plurality of indicators;
detecting a selection input with respect to an indicator of the plurality of indicators;
responsive to said detecting a selection input, retrieving from a memory a treatment protocol from among a plurality of treatment protocols, the treatment protocol corresponding with the indicator and including a number of treatment positions, each treatment position of at least a portion of the number of treatment positions being a position on a recipient where an operation of the therapeutic appliance is specified by the protocol to occur; and
outputting on the display an output including at least a first portion of the treatment protocol.

27. The machine readable medium of claim 26 wherein said operations further comprise obtaining from the protocol a number of operating parameter settings for the treatment appliance, and outputting at least a first operating parameter setting of the number of operating parameter settings.

28. The machine readable medium of claim 27 wherein said operations further comprise detecting a predetermined input and, responsive to said detecting a predetermined input, initiating an operation of the therapeutic appliance in accordance with the at least a first operating parameter setting.

29. The machine readable medium of claim 27 wherein said operations further comprise detecting an override input with respect to the at least a first operating parameter setting, the override input being representative of an alternate value for the first operating parameter setting, detecting a predetermined input and, responsive to said detecting a predetermined input, initiating an operation of the therapeutic appliance in accordance with the alternate value.

30. The machine readable medium of claim 26 wherein said operations further comprise outputting as the at least a first portion of the treatment protocol a visual depiction of a plurality of treatment positions of the number of treatment positions, and identifying at least one of the treatment positions of the plurality of treatment positions as being a current treatment position.

31. The machine readable medium of claim 30 wherein said operations further comprise outputting a visual depiction of a sequential order of the plurality of treatment positions.

32. The machine readable medium of claim 30 wherein said operations further comprise identifying each treatment position of at least a portion of the plurality of treatment positions with a first color, and identifying the current treatment position in a second color that is different than the first color.

33. The machine readable medium of claim 30 wherein said operations further comprise detecting a predetermined input indicative of an operation of the therapeutic appliance and, responsive to said detecting a predetermined input indicative of an operation of the therapeutic appliance, outputting to the display an identification of another treatment position of the plurality of treatment positions as being a current treatment position.

34. The machine readable medium of claim 26 wherein said operations further comprise displaying as the plurality of indicators a plurality of identified anatomical locations.

35. The machine readable medium of claim 34 wherein said operations further comprise outputting a plurality of anatomical categories, each anatomical category of at least a portion of the plurality of anatomical categories having associated therewith a number of anatomical locations of the plurality of anatomical locations, and outputting a depiction of an association between at least a first anatomical category of the at least a portion of the plurality of anatomical categories and the number of anatomical locations associated therewith.

36. The machine readable medium of claim 26 wherein said operations further comprise outputting a visual depiction of a predetermined application member from among a plurality of application members of the therapeutic appliance, the treatment protocol including a specification of the predetermined application member.

37. The machine readable medium of claim 26 wherein said operations further comprise detecting as the selection input a touch input at a position on a touch sensitive overlay of the input apparatus that corresponds with a position on the display where the indicator is depicted.
